(19) United States
(12) Patent Application Publication
(10) Pub. No.: US 2004/0241151 A1
(43) Pub. Date: Dec. 2, 2004

(54) Polymer matrix with lactic acid producing bacteria

(75) Inventors: Ulrika Husmark, Molnlycke (SE); Ingrid Gustafsson, Asa (SE)

Correspondence Address: BURNS DOANE SWECKER & MATHIS L L P, POST OFFICE BOX 1404, ALEXANDRIA, VA, 22313-1404, US

(73) Assignee: SCA Hygiene Products AB, Goteborg (SE)

(21) Appl. No.: 10/851,895

(22) Filed: May 24, 2004

Related U.S. Application Data
(60) Provisional application No. 60/473,192, filed on May 27, 2003.

(51) Int. Cl. A61K 45/00; C12N 1/20

(52) U.S. Cl. 424/93.45; 435/252.9

(57) ABSTRACT

A film-shaped polymer matrix includes lactic acid producing bacteria that are dissolved when exposed to wet conditions. The film-shaped polymer matrix protects bacterial cells from moisture thereby increasing bacterial survival during transport and storage. The film-shaped polymer matrix also results in a high transfer of bacterial cells to the skin of a subject.

### CROSS-REFERENCE TO RELATED APPLICATION

This application claims the benefit of U.S. Provisional Application No. 60/473,192, entitled “Product,” filed on May 27, 2003, the entire contents of which are hereby incorporated by reference.

### BACKGROUND OF THE INVENTION

1. Field of the Invention

The present invention pertains to a film-shaped polymer matrix comprising lactic acid producing bacteria in a film-shaped matrix consisting of one or more polymers that is or are non-toxic and non-irritating to a user's skin and mucous membranes. The invention also pertains to a process for producing such a film-shaped polymer matrix and products containing it.

2. Related Art

The urogenital area harbors a complex microbial ecosystem comprising more than 50 different bacterial species (Hill et al., Scand. J. Urol. Nephrol. 86 (suppl.) 23-29 (1984)). The dominating species for fertile women in this area are lactic acid producing bacteria belonging to the genus Lactobacillus. These lactic acid producing members are important for retaining a healthy microbial flora in these areas, and act as probiotic bacteria with an antagonistic effect against pathogenic microbial species. Lactic acid producing bacteria inhibit growth and colonization by other microorganisms by occupying suitable niches for colonization, by forming biofilms and competing for available nutrients, thereby excluding colonization by harmful microorganisms. Additionally, the production of hydrogen peroxide, specific inhibiting substances, such as bacteriocins, and organic acids (including lactic acid and acetic acid) that lower the pH, inhibit colonization by other microorganisms.

The microbial ecosystem of a healthy individual can be disturbed by the use of antibiotics, during hormonal changes, such as during pregnancy or use of contraceptives with estrogen, during menstruation, after menopause, in people suffering from diabetes, etc. Moreover, microorganisms may spread from the anus to the urogenital area, thereby causing infections. This results in a disturbance of the normal microbial flora and leaves the individual susceptible to microbial infections that cause vaginitis, urinary tract infections, and ordinary skin infections. Microorganisms commonly associated with these kinds of infections belong to the genera Escherichia, Enterococcus, Psedomonas, Proteus, Klebsiella, Streptococcus, Staphylococcus, Gardnerella, and Candida. Women are at particular risk due to the shorter distance between the anus and the urogenital tract. Young women are especially at risk because they do not yet have a well developed microflora in the urogenital area and older women, who no longer have a protective flora.

One way to reduce the problems with the kinds of infections described above is good personal hygiene. However, excessive use of cleaning agents not only decreases the amount of harmful microbes, but can harm the beneficial microbial flora, again rendering it susceptible for pathogenic species to colonize and cause infections. Alternatively, administration of lactic acid producing bacteria to the urogenital area and the skin, in order to outcompete pathogenic species and to facilitate reestablishment and maintenance of a beneficial microbial flora in these areas, has been found to be a successful means to treat and prevent microbial infections.

It has been suggested that lactic acid producing bacteria can be delivered via absorbent products, such as diapers, sanitary napkins, panty liners and tampons, as described in, for example, in WO92/13577, WO97/02846, WO99/17813, WO99/45099 and WO00/35502.

A major problem with providing products intended to be used for transfer of lactic acid producing bacteria, is that the bacteria have to retain viability during transport and storage of the products. Lactic acid producing bacteria rapidly lose viability under moist conditions, and it is therefore important that the bacteria are not exposed to moisture. One way to partly overcome this problem in absorbent products provided with lactic acid producing bacteria has been to supply the products with the bacteria, then drying the products to remove most of the moisture in them and providing the product in moisture impervious packages (WO99/17813). An alternative way to protect bacteria against moisture has been to disperse the bacteria in a hydrophobic substance (see, e.g., U.S. Pat. No. 4,518,696; WO92/13577; WO 02/28446), which, due to its hydrophobic character, will prevent moisture from reaching the embedded bacterial cells.

However, there is still a need to develop alternative ways of protecting lactic acid producing bacteria from moisture that are suitable for the intended administration of the bacteria to a subject and that can be stored for long time periods without loss of viability of the bacterial cells, and that additionally allow efficient transfer of the lactic acid producing bacteria to the user. In addition, there is still a need to develop manufacturing processes that are efficient and less expensive.

### OBJECTS AND SUMMARY

The present inventors have surprisingly found an alternative way to protect lactic acid producing bacterial cells from moisture, thereby increasing bacterial survival during transport and storage. The presented solution also results in a high transfer of bacterial cells to the skin of a subject. According to embodiments of the present invention, the bacterial cells are embedded in a film-shaped polymer matrix which protects the bacteria from moisture, thereby increasing their survival. The invention also relates to a process for producing such a film-shaped polymer matrix comprising lactic acid producing bacteria and products comprising such a film-shaped polymer matrix comprising lactic acid producing bacteria.

### BRIEF DESCRIPTION OF THE FIGURES

FIG. 1 depicts an illustrative example of an absorbent product, such as a sanitary napkin, diaper, panty liner, incontinence guard, and the like, comprising a film-shaped polymer matrix according to an embodiment of the present invention.

FIG. 2 shows a cross-section of the absorbent product depicted in FIG. 1 along the line II-II in FIG. 1.

FIG. 3 depicts a schematic illustration of a tampon comprising a film-shaped polymer matrix according to an embodiment of the present invention.

FIG. 4 shows a cross-section of the absorbent product depicted in FIG. 3 along the line IV-IV in FIG. 3.

FIG. 5 shows the survival of lactic acid producing bacteria in film-shaped polymer matrixes according to the present invention during long term storage.

### DETAILED DESCRIPTION OF THE PREFERRED EMBODIMENTS

The present invention is concerned with the problem of maintaining bacterial viability in products comprising lactic acid producing bacteria from the time for manufacturing until use of the product and obtaining a satisfactory transfer of the bacteria from the product to the user. A factor of major importance for increasing bacterial survival during storage is that the bacteria should be protected from moisture. The present inventors have surprisingly found that embedding the lactic acid producing bacteria in a film-shaped polymer matrix results in greatly enhanced survival of the bacterial cells during storage.

Polymers suitable for the film-shaped polymer matrix protect the bacterial cells from moisture during storage, but are dissolved in bodily fluids and therefore release the bacterial cells when exposed to wet or moist conditions. The film-shaped polymer matrix comprising the lactic acid producing bacteria according to the present invention is composed of at least one polymer that is non-toxic and non-irritating to a user's skin and mucous membranes and at least one lactic acid producing bacterial strain. Polymers suitable for embodiments of the present invention include, but are not limited to, natural hydrophilic polymers, such as polysaccharides and derivatives thereof, such as starch and cellulose (including their derivatives), proteins, hydrophilic polymers, such as synthetic hydrophilic polymers, such as acrylate-based polymers, polyethers, such as polyethyleneoxide, polyurethanes, polyamides, polyacrylnitrile, vinyl-based polymers, such as polyvinyl pyrrolidone and polyvinyl alcohol, etc. Preferred polymers for embodiments of the present invention include, but are not limited to, polyvinyl alcohol, polyethyleneoxide, polyvinyl pyrrolidone and starch. The polymer(s) form a film-shaped matrix that embed the bacteria and thereby protect them from moisture. The polymers can be used alone or in different combinations.

The film-shaped polymer matrix comprising lactic acid producing bacteria according to the present invention may also include additional components. Examples of such components include, but are not limited to, agents protecting the bacteria during the manufacturing of the polymer film, like carbohydrates, such as maltose, sucrose, trehalose, lactose, glucose and fructose, proteins, such as skim milk and albumin, amino acids, such as Na-glutamate, polyols, such as xylitol, mannitol and sorbitol, and antioxidants, such as Na-ascorbate. The majority of these agents may also act as nutrients for bacterial propagation once the polymer film is dissolved. Additional components are also exemplified by plasticizers that can be added to a polymer film comprising starch, like polyhydric alcohols, such as glycerol, polyols, sorbitol and polyvinyl alcohols, sucrose ester, such as sucrose stearate, fatty acids, such as palmitic acid, lipids based on esterified fatty acids, such as monoglycerides, diglycerides and triglycerides, waxes and amino alcohols, such as triethanolamine and N-methyl-diethanol amine, and enzymes, such as amylase.

The concentration of the polymer solution is preferably between 0.1-10% (w/w), more preferably 0.5-7% (w/w), most preferably 1-2% (w/w). The thickness of the film-shaped polymer matrix comprising lactic acid producing bacteria is preferably 50 μm-5 mm, more preferably 100 μm-1 mm, and most preferably 500 μm-1 mm.

In order to increase bacterial survival the water activity of the film-shaped polymer matrix comprising lactic acid producing bacteria is 0.30 or below, preferably 0.25 or below, and most preferably 0.20 or below during at least 3 months storage at 23° C. and 50% relative humidity.

The number of lactic acid producing bacteria in the film-shaped polymer matrix according to the present invention is preferably 10⁷-10¹⁴ CFU/g film and more preferably 10⁹-10¹² CFU/g film.

In order for the film-shaped polymer matrix of a preferred embodiment to allow sufficient transfer of bacteria, the film-shaped polymer matrix preferably dissolves as rapidly as possible when exposed to moist or wet conditions and in either case within 6 hours, preferably within 4 hours.

Lactic acid producing bacteria are chosen for embodiments of the present invention due to their positive effect in preventing and treating microbial infection in the urogenital area and on the skin. The bacteria are preferably isolated from the natural flora of a healthy person, preferably the bacteria are isolated from the skin or urogenital area. Preferred “lactic acid producing bacteria” for the object of the present invention include bacteria from the genera Lactobacillus, Lactococcus and Pediococcus. Preferably, the selected bacteria are from the species Lactococcus lactis, Lactobacillus acidophilus, Lactobacillus curvatus or Lactobacillus plantarum. More preferably, the bacterial strain is selected from Lactobacillus plantarum. Even more preferably, the lactic acid producing bacterium is Lactobacillus plantarum 931 (deposition No. (DSMZ): 11918). The lactic acid producing bacteria can be provided alone or in mixtures containing at least two bacterial strains.

To prevent water vapor from permeating into the hydrophilic film-shaped polymer matrix, thereby interfering with bacterial survival during storage, the film-shaped polymer matrix comprising lactic acid producing bacteria is preferably laminated with a water-vapor barrier material. The survival of the bacterial cells is thereby further increased during storage. Lamination of a film-shaped polymer matrix comprising lactic acid producing bacteria according to embodiments of the present invention also gives a mechanically stronger film that can stand harsher treatment during transport and storage. The film-shaped polymer matrix comprising lactic acid producing bacteria can be laminated on one or both sides. In embodiments where the film-shaped polymer matrix is laminated on both sides, the same laminate composition does not have to be used on both sides. Examples of materials suitable to use for a laminate include, but are not limited to, waxes, wax paper, aluminum foil, polyethylene films, ethylene copolymer, and coextruded films, such as Suranex (Dow Chemical Company, Reinmuenster, Germany). The lamination could be performed using coextrusion technique or by running the film-shaped polymer matrices through rolling, slightly heated cylinders. Optionally, weak adhesives could be used to bond the film-shaped polymer matrix and laminate together. Ultrasonic techniques could also be used for bonding the film-shaped polymer matrix comprising lactic acid producing bacteria and laminate together. Suitable laminate materials have a water vapor transmission rate of, measured according to ASTME 398-83 at 37.8° C. (100° F.) and 90% relative humidity (RH), 10 g/m²/24 h or below, more preferably 5 g/m²/24 h or below, most preferably 2 g/m²/24 h or below. The water-vapor barrier material is to be removed before use of the product comprising the film-shaped polymer matrix.

The manufacturing processes for preparing film-shaped polymer matrices comprising lactic acid producing bacteria according to an embodiment of the present invention involves preparing an aqueous solution of one or more polymers by dissolving the one or more polymers in water, dispersing the bacteria in the aqueous solution of one or more polymers and subsequently drying the dispersion of dissolved polymer and lactic acid producing bacteria on an inert surface at a temperature below 50° C. Optionally, additional components can be added to the polymer solution, either before or after dispersion of the bacteria in the aqueous solution of one or more polymers. The aqueous solution of polymer can comprise one or more polymers. The polymer solution comprising the lactic acid producing bacteria is typically cast onto an inert surface, which can be a laminate material or other inert surface, such as the surface of a hygiene product, using a doctor's blade set to a predetermined width. The cast film-shaped polymer matrices are solidified as the solvent is rapidly evaporated. The evaporation could take place in ambient air, an oven, on a heated roll, by convective drying means or on a surface exposed to IR-radiation. Preferred temperature intervals during evaporation when heated roll or convective drying methods are used are 5-50° C., more preferably 20-40° C. and most preferably 30-37° C. When IR-radiation is used for drying somewhat higher temperatures can be reached without detrimental effects on bacterial survival, due to the short drying times that are required. Typically, drying times in the order of seconds to minutes and drying temperatures up to 65° C. may be used for IR-drying. Thereafter, optionally, the film-shaped polymer matrix comprising lactic acid producing bacteria can be laminated as described above.

The specific growth conditions, harvest conditions and suitable additional components for the lactic acid producing bacteria may be optimized for each specific strain in order to ensure a high survival of the bacteria during manufacturing and storage of the film-shaped polymer matrix. The skilled person is familiar with such optimization. The lactic acid producing bacteria added to the polymer solution can either be freshly prepared or a frozen or dried preparation. Preferably, freshly prepared bacterial preparations are used for embodiments of the present invention.

The dispersion of polymer and lactic acid producing bacteria which can be used for producing a film-shaped polymer matrix according to embodiments of the present invention could also be directly cast or sprayed onto a material, or a material can be dipped in the dispersion of polymer and lactic acid producing bacteria, which after drying, is a part of a product, such as a hygiene product.

There are other processes available for the formation of film-shaped polymer matrixes comprising lactic acid producing bacteria, i.e., calendering and extrusions that can be used as long as they are operating at temperatures not harmful for the bacteria.

When starch is used for manufacturing of the film-shaped polymer matrix, the starch may be gelatinized before use. One way this might be performed is by heating a suspension of the starch (grains or granules) in water at a temperature of approximately 90-100° C. for 30-60 min. Before addition of the bacteria to the gelatinized starch, the suspension is preferably cooled to 37° C. or less.

The film-shaped polymer matrix comprising lactic acid producing bacteria according to an embodiment of the present invention is preferably added to hygiene products, such as hygiene tissues, incontinence guards, diapers, panty liners, tampons, sanitary napkins, etc., in which the film-shaped polymer matrix comprising lactic acid producing bacteria, when exposed to moisture and wet conditions, results in dissolution of the film-shaped polymer matrix and transfer of the lactic acid producing bacteria to the skin and/or the urogenital area.

By “hygiene tissue” is meant any device for wiping skin, for instance, a washcloth, patch, towelette, napkin, wetwipe, and the like. The hygiene tissue provided can be composed of a matrix comprising any natural or synthetic fiber, such as rayon, cellulose, regenerated cellulose, polyester, polyolefine fibers, textile and the like, or foam, nonwoven, felt or batting, or combinations thereof. The film-shaped polymer matrix comprising the lactic acid producing bacteria is applied to the hygiene tissue by dipping the tissue in a dispersion of polymer and lactic acid producing bacteria which can be used for producing a film-shaped polymer matrix before drying of the hygiene tissue.

The film-shaped polymer matrix comprising lactic acid producing bacteria according to embodiments of the present invention, is, as described above, particularly suitable for application to absorbent products, such as sanitary napkins, panty-liners, diapers, tampons, incontinence guards, etc., because these products provide a convenient means for delivery of lactic acid producing bacteria to the urogenital area. The absorbent products according to embodiments of the invention are preferably composed of a liquid-permeable casing sheet, a liquid-impermeable backing sheet, an absorbent layer, comprised of one or more layers, placed between said upper layer and said back sheet and optionally a device for adherence. The film-shaped polymer matrix according to an embodiment of the present invention is preferably placed on the permeable casing sheet, but can also be placed inside the

casing sheet. Alternatively, the film-shaped polymer matrix comprising lactic acid producing bacteria according to the present invention can be provided separately and placed on the absorbent product by the users before use. The number of probiotic bacteria in an absorbent product according to the present invention is 10⁷-10¹⁴ CFU, preferably 10⁸-10¹¹ CFU, most preferably 10⁹-10¹⁰ CFU.

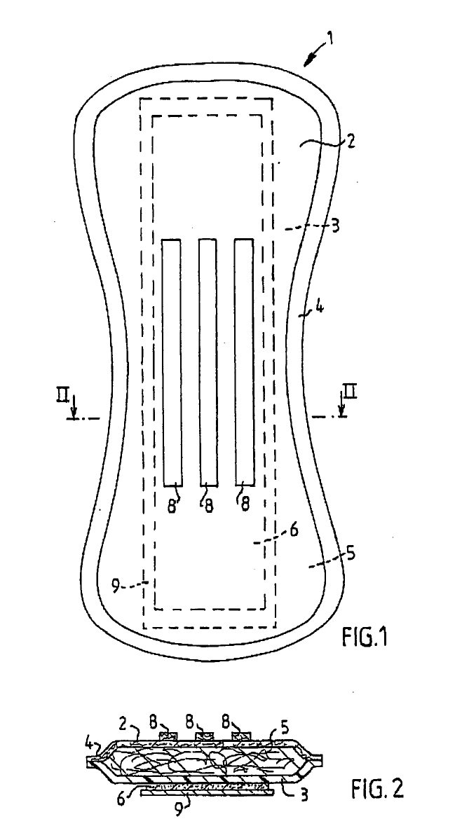

A more detailed description of an absorbent product, such as a sanitary napkin, panty liner, diaper, or incontinence guard is given below. The absorbent product 1 shown in FIG. 1 and FIG. 2 (cross-section of the absorbent product depicted in FIG. 1 along the line II-II in FIG. 1) includes a liquid-permeable casing sheet or top sheet 2 disposed on that side of the absorbent product which is intended to lie proximal to the wearer in use. In one embodiment, the liquid-permeable casing sheet 2 will conveniently comprise a somewhat soft, skin-friendly material. Different types of non-woven material are examples of suitable liquid-permeable materials. Other casing sheet materials that can be used include, but are not limited to, perforated plastic films, net, knitted, crocheted or woven textiles, and combinations and laminates of the aforesaid types of material.

The absorbent product 1 also includes a liquid-impermeable casing sheet or backing sheet 3, disposed on that side of the napkin 1 distal from the wearer in use. The liquid-impermeable casing sheet 3 is conventionally comprised of thin plastic film. Alternatively, there may be used a liquid-permeable material that has been rendered impermeable to liquid in some way or another. For instance, the liquid-permeable material may be coated with a glue that is impermeable to liquid, and the liquid-permeable layer laminated with a liquid-impermeable material, or hot-calendering a material that was initially liquid-permeable, such as to melt down the surface of the material and therewith obtain a liquid-impermeable layer. Alternatively, there may be used other textiles comprised of hydrophobic fibers and so impervious as to enable them to be used as a liquid barrier layer. The liquid-impermeable casing sheet 3 may beneficially be vapor-permeable.

The two casing sheets 2, 3 form a joining edge 4 that projects outwardly around the napkin contour line, and are mutually joined at this edge. The sheets may be joined together by means of any appropriate conventional technique, such as gluing, welding, or sewing.

The absorption core 5 sandwiched between the casing sheets 2, 3 may constitute the layer capable of receiving and storing essentially all liquid discharged by the wearer. The absorption core 5 may, for instance, be produced from cellulose pulp. This pulp may exist in rolls, bales or sheets that are dry-defibered and converted in a fluffed state to a pulp mat, sometimes with an admixture of superabsorbents, which are polymers capable of absorbing several times their own weight of water or body liquid (fluid). Examples of other usable materials are different types of foamed materials known, for instance, from SE 9903070-2, natural fibers, such as cotton fibers, peat, or the like. It is, of course, also possible to use absorbent synthetic fibers, or mixtures of natural fibers and synthetic fibers. patent application SE 9903070-2 describes a compressed foam material of regenerated cellulose, e.g., viscose. Such foam material will preferably have a density of 0.1 to 2.0 g/cm³. The absorbent material may also contain other components, such as foam-stabilizing means, liquid-dispersing means, or a binder, such as thermoplastic fibers, for instance, which have been heat-treated to hold short fibers and particles together so as to form a coherent unit.

A fastener means 6 in the form of an elongate rectangular region of self-adhesive is provided on the surface of the liquid-impermeable casing sheet 3 that lies distal from the wearer in use. The fastener means 6 extends over the major part of the liquid-impermeable casing sheet 3. Embodiments of the invention are not restricted to the extension of the fastener means 6, and the means may have the form of elongate stripes, transverse regions, dots, circles, or other patterns and configurations. Neither are embodiments of the invention restricted to the use of solely adhesive fastener means, since friction fasteners may be used and other types of mechanical fasteners, such as press studs, clips, girdles, pants or the like may be used when found suitable to do so. When an adhesive fastener is used this is commonly protected, by a protective layer 9, from adhering to other surfaces prior use, which would destroy the fastener means.

The film-shaped polymer matrix comprising lactic acid producing bacteria according to embodiments of the present invention, when used in an absorbent product, is arranged onto or directly beneath the liquid-permeable casing sheet 2. Preferably, the film-shaped polymer matrix comprising lactic acid producing bacteria is placed in such a way that the film-shaped polymer matrix does not cover the entire surface of the absorbent product. In this way, the film-shaped polymer matrix comprising lactic acid producing bacteria does not interfere with absorption of bodily fluids (such as blood, urine, secretion, etc.) by the absorbent product. Even if the film-shaped polymer matrix comprising lactic acid producing bacteria solubilizes by bodily fluids, initial absorption by the absorbent product can be impaired before the film-shaped polymer matrix is solubilized if the whole of an absorbent product is covered by the film-shaped polymer matrix. One way to solve this is to supply the absorbent article with a film-shaped polymer matrix comprising lactic acid producing bacteria according to an embodiment of the present invention which has been punched, forming at least one opening through which bodily fluids can be transported. More preferably, a carrier in the form of a net or loose non-woven sheet, dipped in a dispersion of polymer and lactic acid producing bacteria which can be used for producing a film-shaped polymer matrix according to the present invention, may also be arranged onto or directly beneath the liquid-permeable casing sheet. In FIGS. 1 and 2, one way to place the film-shaped polymer matrix comprising lactic acid producing bacteria is exemplified, i.e., the film-shaped polymer matrix is placed in stripes 8.

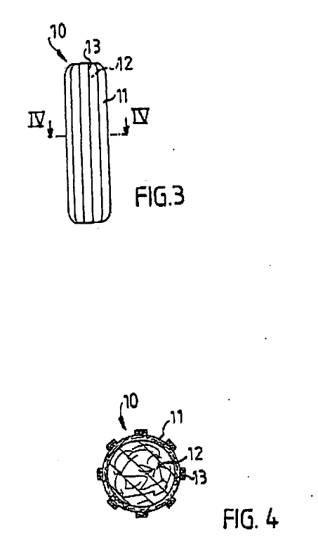

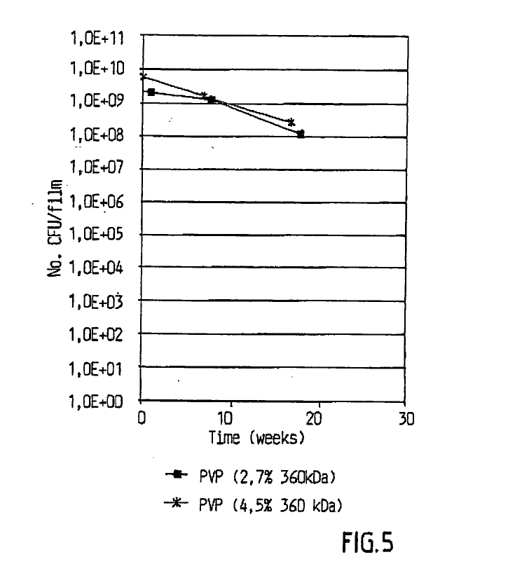

In a similar manner to what is described above, a tampon comprising the film-shaped polymer matrix can be prepared. FIG. 3 and 4 (cross-section of the tampon in FIG. 3 along the line IV-IV) depict a schematic exemplary drawing of a tampon 10 comprising a film-shaped polymer matrix 13 according to the present invention, wherein the film-shaped polymer matrix 13 is arranged onto the casing sheet 11. Also depicted is the absorbent core 12.

The skilled person could readily use the above exemplary descriptions of hygiene products comprising a film-shaped polymer matrix according to an embodiment of the present invention to manufacture any hygiene product comprising a film-shaped polymer matrix according to an embodiment of the invention. Therefore, alternative designs of a sanitary napkin, incontinence guard, panty-liner, diaper, tampon, hygiene tissue, etc., are also included within the present invention.

The embedding of lactic acid producing bacteria in a film-shaped polymer matrix according to an embodiment of the present invention is also suitable for increasing the survival of the bacteria in pharmaceutical preparations and in the food industry.

A film-shaped polymer matrix comprising lactic acid producing bacteria according to embodiments of the present invention has several advantages. When freeze-dried bacteria or suspensions of bacteria are added directly to a hygiene product, the product has to be dried in order for the inherent moisture content in the product not to affect bacterial survival negatively. This is both a complicated process, because the whole product has to be dried, but drying of the hygiene product can also result in a lowered initial absorption of bodily fluids as some residual moisture facilitates initial absorption. In addition, further protection from moisture during transport and storage by water-impervious packaging is necessary for maintenance of bacterial survival in this case. This necessity to provide the whole hygiene product in a moisture-impervious packing unit results in higher production costs. In comparison, by embedding the bacteria in a film-shaped polymer matrix according to an embodiment of the present invention, one avoids the need for drying the hygiene product and the use of moisture impervious packing units, with high survival of the bacterial cells, even after prolonged storage. The use of a film-shaped polymer matrix according to embodiments of the present invention furthermore alleviates the use of freeze-dried bacterial preparations, which are the mostly common preparation form for bacteria for use in hygiene products, but which are costly and complicated to prepare. Instead, suspensions of lactic acid producing bacteria can be used directly when preparing a film-shaped polymer matrix comprising lactic acid producing bacteria which is cheaper and more practical.

A film-shaped polymer matrix comprising lactic acid producing bacteria can also be prefabricated and later placed on many different products, such as hygiene products etc. without any special adaptations for the different products. Also, the production of products comprising bacteria requires special hygiene requirements at the manufacturing plant. Costs can therefore be reduced by producing the film-shaped polymer matrix comprising lactic acid producing bacteria at another location.

Using a film-shaped polymer matrix in order to protect lactic acid producing bacteria from moisture leads to high stability at common temperatures during transport and storage, since the film structure itself is very insensitive to temperature variations. Also, the dryness of the bacterial cells in the dry film-shaped polymer matrix renders the bacteria more heat-tolerant.

Furthermore, the preparation of a film-shaped polymer matrix is more gentle to the lactic acid producing bacteria, compared to, for example, extrusion or spraying that is often used when the bacteria are mixed with hydrophobic substances.

The use of a film-shaped polymer matrix comprising lactic acid producing bacteria is also advantageous in terms of efficacy of transfer of the bacteria to the skin. When the film-shaped polymer matrix comprising lactic acid producing bacteria dissolves, fragments of the film-shaped polymer matrix may be transferred to the skin where they dissolve further. The film fragments thereby act as a vehicle for transfer of the bacteria. Additionally, the use of a film-shaped polymer matrix ensures that the bacteria are kept in the outer layers of the hygiene product, thereby ensuring a high transfer rate. When the film-shaped polymer matrix comprising lactic acid producing bacteria is applied to a hygiene product, transfer rates can also be optimized by the choice of commonly used surface materials.

The present invention therefore solves many of the problems associated with providing products comprising lactic acid producing bacteria. Below the present invention is further described by illustrative but non-limiting examples.

### EXAMPLES

### Example 1

Production of a film-shaped polymer matrix comprising lactic acid producing bacteria.

An aqueous polymer solution with a concentration of 0.1-10% by weight is prepared by dissolving polyethylene oxide, polyvinyl pyrrolidone, polyvinyl alcohol or starch in water.

One part of bacterial suspension (ca 10¹⁰ CFU/ml, BioNativ AB, Box 7979, 907 19 Umeå, Sweden) comprising Lactobacillus plantarum 931 (deposition No. (DSMZ): 11918) is mixed with 9 parts of the polymer solution for 5 minutes. The mixture is poured into small Petri dishes in a quantity that ensures the right thickness and an amount of bacteria of approximately 10⁹ cfu/film-shaped polymer matrix. The resulting film-shaped polymer matrix comprising lactic acid producing bacteria has a thickness of preferably 50 μm-5 mm, more preferably 100 μm-1 mm and most preferably 500 μm-1 mm.

The petri dish is placed in a climate chamber at a temperature of 37° C. and with as low relative humidity as possible (10% or below), whereby the water evaporates and the film-shaped polymer matrix solidifies and the bacteria are immobilized in the film-shaped polymer matrix.

The water activity of the film-shaped polymer matrixes comprising lactic acid producing bacteria is measured using an a_{w}-instrument; DD401102 Aqualab Serie 3TE (ADAB Analytical Devices AB, Stockholm, Sweden).

Within 1-2 weeks after production of the film-shaped polymer matrix transfer tests from absorbent products comprising the film-shaped polymer matrix according to the present invention to skin are performed (see below).

### Example 2

Survival of L. plantarum 931 in film-shaped polymer matrices according to an embodiment of the present invention.

The film-shaped polymer matrix is placed in a climate chamber at 23° C. and 50% relative humidity. The survival of the bacteria is tested in a film-shaped polymer matrix according to Example 1 further coated with Caremelt (a mixture of waxes, Cognis, Henkel KgaA, Dusseldorf, Germany). The survival of the bacteria in the film-shaped polymer matrix is tested at predetermined intervals for several months (see below).

To test the survival of the lactic acid producing bacteria in the film-shaped polymer matrix, the film is placed in a petri dish, immersed with 20 ml of NaCl (0.85%) and put on a shaking-device. After 40 minutes, the film-shaped polymer matrix is dissolved and the survival of the bacteria is determined by counting the number of colony forming units (CFU) by standard spread-plate techniques and cultivation on MRS agar (2 days of incubation at 37° C.). The results are presented in FIG. 5. PVP indicates a polyvinyl pyrrolidone matrix.

### Example 3

Transfer of L. plantarum 931 from a panty-liner provided with a film-shaped polymer matrix.

Film-shaped polymer matrices comprising lactic acid producing bacteria are produced as described in Example 1. About 1 cm² of film-shaped polymer matrix are cut out, weighed and placed on the nonwoven top layer of a panty-liner specimen (a circle 2.5 cm in diameter, punched out of an absorbent product).

100 μl of NaCl are added with a pipette to the absorbent product, comprising the film-shaped polymer matrix with bacteria, and the specimen is subsequently mounted, with constant pressure (elastic tape and elastic bandage), onto the forearm of volunteers. After 2 hours, the product is removed and the number of transferred Lactobacilli on the skin measured. A sterile stainless-steel cylinder (2.6 cm in diameter, height 2 cm) is held tight to the skin (that has been covered with the specimen), and 1 ml of phosphate buffer (0.1 M, pH 7.2) is poured into the cylinder. With a smooth glass stick, the skin is gently “kneaded” for 1 minute. Afterwards, the buffer is collected with a pipette and CFU measured with spread-plate technique and MRS agar.

The percentage of transferred Lactobacilli is calculated by dividing the number of CFU collected from the skin area covered by the specimen with the total number of CFU in the film-shaped polymer matrix on the test specimen. The number of Lactobacilli initially present on the skin at the sample site is very low, especially with respect of the number of L. plantarum 931 transferred to the skin. Therefore, the number of Lactobacilli detected on the skin after the transfer test are considered to be a result of transfer from the specimen comprising L. plantarum 931. As a comparison, a panty liner with a dry bacterial preparation not embedded in a film-shaped polymer matrix was used. As can be seen in Table 1, the percentage of bacteria transferred to the skin from an absorbent product was enhanced using a film-shaped polymer matrix according to an embodiment of the present invention.

TABLE 1

| | | | No. of CFU transferred | Initial No. of | |
|---|---|---|---|---|---|
| Polymer | Conc. (w/w) | MW (kDa) | to the skin | CFU on product | Transferred (%) |
| PVOH | 1 | 96 | 3.E+04 | 1.E+06 | 2.4 |
| PVP | 1 | 40 | 7.E+07 | 6.E+08 | 12.7 |
| Comparison | | | 2.E+05 | 4.E+07 | 0.4 |

PVP = Polyvinyl pyrrolidone
PVOH = Polyvinyl alcohol

Although only preferred embodiments are specifically illustrated and described herein, it will be appreciated that many modifications and variations of the present invention are possible in light of the above teachings and within the purview of the appended claims without departing from the spirit and intended scope of the invention.

What is claimed is:

1. A film-shaped polymer matrix comprising at least one lactic acid producing bacterial strain in one or more polymers which is non-toxic and non-irritating to a user's skin and mucous membranes, wherein the one or more polymers is able to protect bacterial cells from moisture during storage and is dissolvable by bodily fluids.

2. The film-shaped polymer matrix of claim 1, wherein the at least one lactic acid producing bacterial strain is isolated from the skin or urogenital area of a healthy person.

3. The film-shaped polymer matrix of claim 1, wherein the at least one lactic acid producing bacterial strain is selected from the genera Pediococcus, Lactococcus, Lactobacillus or a mixture thereof.

4. The film-shaped polymer matrix of claim 3, wherein the lactic acid producing bacterial strain is Lactobacillus plantarum.

5. The film-shaped polymer matrix of claim 4, wherein the lactic acid producing bacterial strain is Lactobacillus plantarum 931 (deposition No. (DSMZ): 11918).

6. The film-shaped polymer matrix of claim 1, wherein the one or more polymers is selected from the group consisting of polysaccharides and derivatives thereof and synthetic hydrophilic polymers and derivatives thereof.

7. The film-shaped polymer matrix of claim 1, wherein the one or more polymers is selected from the group consisting of polyvinyl alcohol, polyethyleneoxide, polyvinyl pyrrolidone, and starch.

8. The film-shaped polymer matrix of claim 1, further comprising at least one additional component.

9. The film-shaped polymer matrix of claim 1, wherein the film-shaped polymer matrix has a thickness of 50 μm-5 mm.

10. The film-shaped polymer matrix of claim 1, wherein the water activity of the film-shaped polymer matrix is 0.30 or below.

11. The film-shaped polymer matrix of claim 1, wherein the bacterial concentration is 10⁷-10¹⁴ colony forming units (CFU)/g film.

12. The film-shaped polymer matrix of claim 1, further comprising a laminate layer placed on at least one side of the film-shaped polymer matrix.

13. A hygiene product comprising a film-shaped polymer matrix comprising lactic acid producing bacteria of claim 1.

14. A process for producing a film-shaped polymer matrix of claim 1 comprising lactic acid producing bacteria comprising:
a) preparing an aqueous solution of one or more polymers that is non-toxic and non-irritating to a user's skin and mucous membranes;
b) dispersing lactic acid producing bacteria in said solution of the one or more polymers;
c) optionally, adding at least one additional component to the dispersion;
d) drying said resulting dispersion comprising lactic acid producing bacteria on an inert surface, at a temperature below 50° C., thereby producing a film-shaped polymer matrix; and
e) optionally, laminating the resulting film-shaped polymer matrix; wherein steps b) and c) can be performed in any order.

15. The process of claim 14, wherein the concentration of the polymer solution is between 0.1-10% (w/w).

16. The process of claim 14, wherein the thickness of the resulting film-shaped polymer matrix is between 50 μm-5 mm.

17. The process of claim 14, wherein the concentration of bacteria in the film-shaped polymer matrix is 10⁷-10¹⁴ CFU/g film.

18. The process of claim 14, wherein the film-shaped polymer matrix is laminated on one or both sides.

19. The process of claim 14, wherein the film-shaped polymer matrix is laminated by coextrusion; running the polymer matrix through rolling, heated cylinders; by bonding the polymer matrix and a laminate with adhesive; or by ultrasonic bonding.

20. A kit comprising
a) a hygiene product; and
b) a film-shaped polymer matrix of claim 1.
